(19) United States
(12) Patent Application Publication
(10) Pub. No.: US 2021/0298591 A1
(43) Pub. Date: Sep. 30, 2021

(54) TRANSLUCENT ILLUMINATED ENDOSCOPIC PROBE

(71) Applicant: OPTICAL SPINE, LLC, Grand Blanc, MI (US)

(72) Inventors: Avery M. JACKSON, III, Grand Blanc, MI (US); John Souza, SR., Monroe, NC (US)

(73) Assignee: OPTICAL SPINE, LLC, Grand Blanc, MI (US)

(21) Appl. No.: 17/266,718

(22) PCT Filed: Aug. 9, 2019
(86) PCT No.: PCT/US2019/045956

§ 371 Date: Feb. 8, 2021

Related U.S. Application Data
(60) Provisional application No. 62/716,677, filed on Aug. 9, 2018.

(51) Int. Cl. A61B 1/317; A61B 17/34; A61B 1/06; A61B 1/00; A61B 1/04

(52) U.S. Cl. CPC A61B 1/317; A61B 17/3472; A61B 1/0669; A61B 2017/00902; A61B 1/042; A61B 17/3421; A61B 2017/3454; A61B 1/00066

(57) ABSTRACT

An illuminated translucent endoscopic probe system includes a handle and rigid elongate body for passage into and through hard and/or bony tissue and includes an integrated camera and light source positioned distally from the handle and adjacent a distal tip that includes a transparent driving head. The system provides real time visualization at the distal end as it passes into and through tissue.

### RELATED APPLICATIONS

This application claims the benefit under 35 U.S.C. § 119(e) to U.S. Provisional Application Ser. No. 62/716,677 filed Aug. 9, 2018 and PCT Application No. PCT/US2019/045956 filed on Aug. 9, 2019, the entirety of which applications are incorporated herein by reference.

### BACKGROUND

Placement of instruments and implants within tissue in a body can present a variety of challenges. Whether penetration of bone is required, or penetration of varying types of soft tissue is required, there is typically a need for visualization. There are many examples of laparoscopic devices and systems that provide visualization once entry into the tissue has been accomplished. But there is a lack of technology available for allowing entry into the tissue with visualization in real time beyond the context of intraluminal systems, such as for the gastrointestinal system and vasculature. In particular, there are scant options for visualization while penetrating bone, or for penetrating soft and semi-soft tissue and connective tissue between organs and bones. One such example includes penetration of bone for placement of screws, such as penetration of the pedicle in a spinal vertebra. Another example is penetration into Kambin's triangle for transforaminal access to intervertebral discs through the foramen that allows preservation of paravertebral muscles and the facet joint.

With regard to the first example where visualization would be beneficial, penetration of the pedicle of a vertebra is needed for most spinal fusion surgeries that do not involve anterior access to the spine. The pedicle is a dense, stem-like structure projecting from the posterior of a vertebra, and there are two pedicles per vertebra that connect to other structures. Since the pedicle is the strongest point of attachment of the spine, significant forces can be applied to the spine without failure of the bone-to-metal connection. To insert pedicle screws, a long, thin, metal probe is inserted through the pedicle and into the vertebral body, forming a hole for reception of the screw. Conventional pedicle probes may be straight or curved and comprise an elongate solid metal shaft with an enlarged hand grip on the proximal end. The probe may have a shaped distal end adapted for forming a hole through the pedicle, or a separate awl or reamer may first be used to form a hole through the pedicle, and the probe then inserted into the cancellous bone of the pedicle and into the vertebral body to develop a path for the screw. A variety of probes are known in the prior art, including the so-called gear shift pedicle probe and the Fox pedicle probe. The gear shift probe has a round head on its proximal end, whereas the Fox probe has a flat disc-shaped head on its proximal end.

Conventional modalities used to approximate or simulate screw placement are indirect and include fluoroscopic guidance and frameless stereotactic guidance. Approximations of the pedicle and surrounding vital structures are obtained from a CT scan or Mill done prior to surgery. Proper positioning of a conventional probe depends to an extent upon tactile feel. For instance, advancement of the probe should be smooth and consistent. A sudden plunge suggests breaking out of the pedicle laterally, and an increase in resistance indicates abutment against the pedicle or vertebral body cortex. These conventional modalities require a steep learning curve, and improper or inaccurate manipulation of the probe and placement of the pedicle screw can result in caudal or medial penetration of the pedicle cortex and dural or neural injury. In addition, for conventional pedicle probes there is no direct way to confirm that the hole was made within the pedicle and that the screw will be placed completely inside the pedicle. Surrounding structures can be injured if a portion of the screw is placed outside of the pedicle. There can be nerve root injury, epidural vessel injury, or spinal fluid leakage caused by a misplaced screw.

Accordingly, there is need for a system and method for insertion of pedicle screws which eliminates the guesswork and error-prone modalities of the prior art and provides confirmation during the surgical procedure that the pedicle probe is in the right position for forming a hole for proper placement of the pedicle screw.

Further, with regard to the second example for accessing tissue via Kambin's triangle, though the tissue is not as resistant as pedicle bone, it does present resistance that is too great for flexible scope systems and it is not suited for use of more conventional laparoscopes, any of which are useful only once access to the target tissue is achieved. Thus, there is a need for a system and method that enables visualization while the tissue is being penetrated and is capable of penetrating the tissue while guiding the surgeon to steer clear of nerves and other sensitive structures. More generally, there is need for an endoscopic system which provides a surgeon with direct visualization in real time of a site within the body, particularly within passages formed through solid tissue such as bone, and in void spaces within or adjacent bony tissue.

### SUMMARY

This invention relates generally to surgical instruments. More specifically, the invention relates to an tissue penetrating translucent illuminated endoscopic probe for use in tissue for visualization within the tissue in real time. In some examples, visualization may be achieved using an inventive tissue penetrating translucent illuminated endoscopic probe according to the disclosure within any one or more anatomical spaces, for example including, an open space in tissue, a passage in a bone (after or while the passage is being formed), within a disc space between two vertebra, within a joint articular space, within other solid tissue other than bone, in a space such as Kambin's triangle (a three-dimensional anatomical right triangle located over the dorsolateral intervertebral disc of the lumbar spine and a common access passage for reaching the spine), and in other anatomical spaces.

In some particular embodiments, the invention relates to an tissue penetrating translucent illuminated endoscopic probe (“probe”) for passage through generally solid tissue, such as bone, and in some particular uses, for passage through bone, such as a vertebral pedicle, in preparation for pedicle screw insertion. In some embodiments, the invention relates to a probe that is able to penetrate cortical bone, or to advance through cancellous bone, or combinations of these. In yet other embodiments, the probe is adapted to penetrate tissue such as muscle and connective tissue without the need for cutting and/or cauterizing instruments.

In one embodiment, provided is an illuminated translucent endoscopic probe system that includes a handle and rigid elongate body for passage into and through hard and/or bony tissue and includes an integrated camera and light source positioned distally from the handle and adjacent a distal tip that includes a transparent driving head. The probe system includes slidably engageable components, one or more of which include proximal handles that can be operated independently or can be engagable for coordinated operation. In another embodiment, the probe includes a proximal handle and a distal tapered tip and a body between the handle and the tip.

According to one feature, the probe incorporates at least one light source to enable a user of the probe to see the area into which the probe tip is inserted. And the probe incorporates at least one camera positioned either proximally or distally within the probe body. in some specific embodiments the camera and the lighting are positioned distally and are either central within the probe at its elongate axis, or oriented laterally from the axis and directed towards the distal end to enable real time visualization at the distal tip as the probe is moved forward in tissue. According to the embodiments, the camera and light are integrated and positioned distally adjacent a transparent driving head within the penetrating probe. The transparency of the driving head is sufficient to enable the passage of light from a light source to direct and provide illumination at the distal end and visualization through the camera. In some embodiments, transparency along at least a portion of the probe and a retainer for one or more cameras positioned at location that is proximal to and above the distal tip can provide additional visibility of the probe and surrounding tissue at the probe's distal end. According to other features, the probe optionally may include any one or more of a removable tip on its distal end, a distal cutting feature for reaming into bone, and one or more of irrigation and suction capabilities.

### BRIEF DESCRIPTION OF THE DRAWINGS

The foregoing as well as other objects and advantages of the invention will become apparent from the following detailed description when considered in conjunction with the accompanying drawings, wherein like reference characters designate like parts throughout the several views, and wherein:

FIG. 1 is a schematic perspective view of an inventive illuminated translucent endoscopic probe according to the instant disclosure, represented in the context of a vertebral bone and with specific reference to the pedicle (P);

FIG. 2 is a side view in elevation of the inventive illuminated translucent endoscopic probe depicted in FIG. 1, wherein a portion of the probe is shown in close-up relative to FIG. 1;

FIG. 3 is a side cross sectional perspective view of the inventive illuminated translucent endoscopic probe shown in FIG. 2, the cross-sectional view along the longitudinal axis of the probe;

FIG. 4 is a cross sectional view across the longitudinal axis in the body of the inventive illuminated translucent endoscopic probe showing the center and peripheral voids;

FIG. 5 is a cross-sectional view across the longitudinal axis through the body of in alternate embodiment of the inventive illuminated translucent endoscopic probe showing the center and peripheral voids;

FIG. 6 is a cross-sectional view across the longitudinal axis in the tip portion of the inventive illuminated translucent endoscopic probe showing the central through channel;

FIG. 7 is a schematic perspective view of an inventive tissue penetrating translucent illuminated endoscopic probe system according to the instant disclosure, represented in the context of a vertebral bone and with specific reference to the pedicle;

FIG. 8 is an exploded perspective view of the inventive probe system depicted in FIG. 7;

FIG. 9 is a perspective view of the outer access cannula component of the inventive probe system depicted in FIG. 7;

FIG. 10 is a perspective view of the outer penetrating cannula component of the inventive probe system depicted in FIG. 7;

FIG. 11 is a perspective view of the scope with integrated camera and light component of the inventive probe system depicted in FIG. 7; and

FIG. 12 is a cross-sectional view across the longitudinal axis in the distal tip portion of the inventive probe system depicted in FIG. 7;

FIG. 13 is an enlarged exploded perspective view of the penetrating cannula and transparent driving head components of the inventive probe system depicted in FIG. 7;

FIG. 14 is an enlarged perspective view of an alternate embodiment of a penetrating cannula and transparent driving head component of the inventive probe system;

FIG. 15 is an enlarged cross-sectional view across the longitudinal axis in the distal tip portion of the inventive probe system depicted in FIG. 14;

FIG. 16 is an enlarged side perspective view of the outer access cannula, penetrating cannula and transparent driving head portions of the inventive probe system depicted in FIG. 14;

FIG. 17 is an enlarged perspective view of the distal tip of the scope with integrated camera and light;

FIG. 18 is an enlarged perspective view of an alternated embodiment of a penetrating cannula and transparent driving head components of the inventive probe system;

FIG. 19 is a cross-sectional view along the longitudinal axis in the distal tip portion of the inventive probe system depicted in FIG. 18; and

FIG. 20 is an enlarged side perspective view of the outer access cannula, penetrating cannula and transparent driving head portions of the inventive probe system depicted in FIG. 18.

### DETAILED DESCRIPTION

In accordance with the instant disclosure, an tissue penetrating translucent illuminated endoscopic probe is provided that generally conforms in its basic features with conventional probes, such as probes for penetrating bony tissue. The illuminated translucent endoscopic probe thus includes conventional features that include a handle, a body and a penetrating tip. A representative conventional probe, for frame of reference, is a conventional Fox pedicle probe that has a disc-shaped head on its proximal end that is about two inches in diameter, and an elongate solid metal shaft projecting from the center of one side thereof. A reduced diameter tip on the distal end of the shaft is configured to act as a reamer, i.e., it may have a fluted configuration as found on drill bits. In use, a surgeon places the disc-shaped head in the palm of his or her hand, with the shaft extending forwardly. The tip is then pushed against the pedicle while the probe is being rotated back and forth about the longitudinal axis of the shaft to form a hole in the pedicle for reception of a pedicle screw.

According to the various embodiments, the instantly disclosed tissue penetrating translucent illuminated endoscopic probe system includes features that enable real time visualization directly at the distal end of the tissue penetrating translucent illuminated endoscopic probe system as it is moved through tissue. It will be appreciated that while the instant disclosure provides by way of example only use of the inventive tissue penetrating translucent illuminated endoscopic probe system in the context of surgical procedures in the spine, and specifically accessing the pedicle of a vertebrae, the instant disclosure extends to use in any other tissue in which a space within the body is accessed for surgical examination or intervention. In some particular embodiments, the instant disclosure provides an tissue penetrating translucent illuminated endoscopic probe system which is particularly suited for passage through and visualization in solid tissue and in particular bony tissue. In some other examples, the tissue penetrating translucent illuminated endoscopic probe system may be used to visualize within a disc space between two vertebra, within a joint articular space, within other solid tissue other than bone, in a space such as Kambin's triangle (a three-dimensional anatomical right triangle located over the dorsolateral intervertebral disc of the lumbar spine and a common access passage for reaching the spine), and in other anatomical spaces.

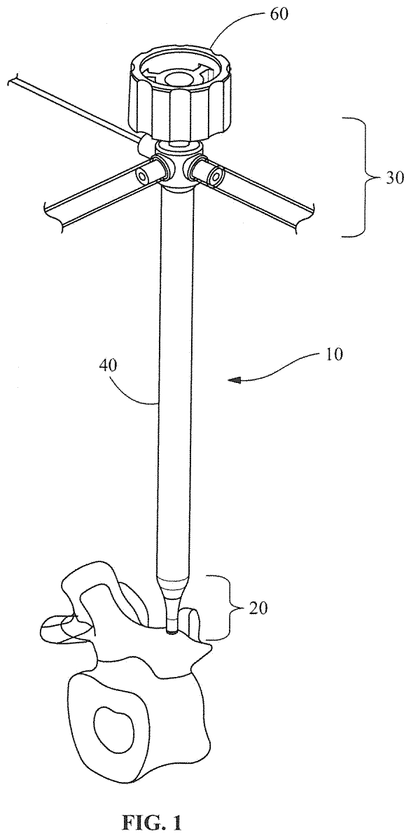

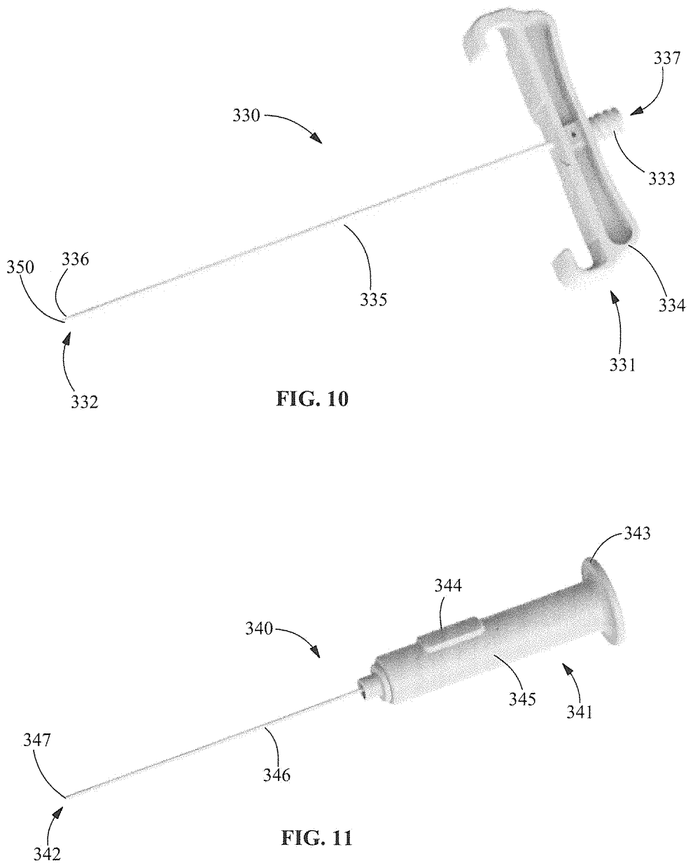

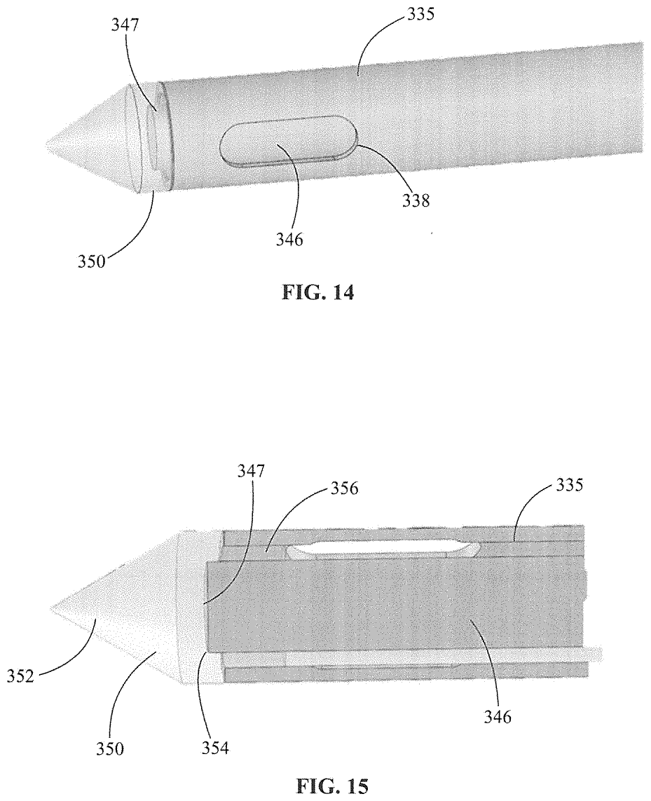

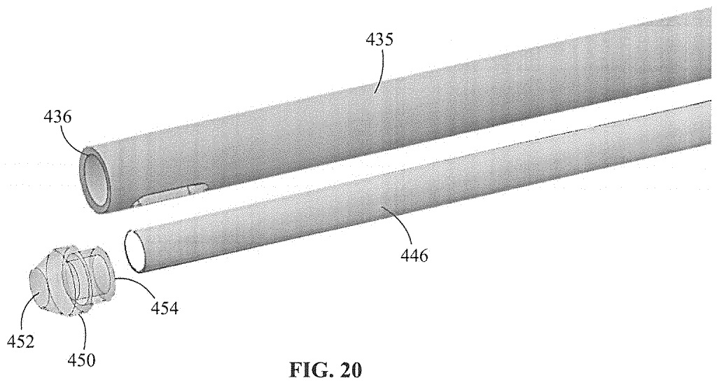

FIGS. 1-20, which are now referenced, illustrate various embodiments of the present invention and the manner in which they are assembled each having like reference numerals refer to like components according to the drawing and the key to reference numerals provided herein.

Referring now to the drawings, in some embodiments, an inventive illuminated translucent endoscopic probe according to the disclosure is shown in FIG. 1-FIG. 6.

As shown in FIG. 1, in some embodiments, the inventive illuminated translucent endoscopic probe 10 is formed at least in part of a transparent polymeric, transparent glass or glass like material. In particular, at least a portion of the illuminated translucent endoscopic probe 10 is formed of such a transparent polymeric, transparent glass or glass like material. As further described herein below, the illuminated translucent endoscopic probe 10 incorporates at least one light source to enable a user to see the area into which the probe is inserted. And the illuminated translucent endoscopic probe 10 incorporates at lighting and least one camera positioned proximally to enable real time visualization as the probe is moved forward in tissue. The probe is transparent along at least a portion of its length, the transparency sufficient to enable the passage of light from a proximally positioned light source to direct and provide illumination at the distal end 22. Likewise, the probe transparency along at least a portion of the probe is sufficient to enable one or more cameras positioned at location that is proximal and above the distal end 22 to provide an image thereof and of surrounding tissue. According to other features, the probe optionally may include any one or more of a removable tip on its distal end, a distal cutting feature for reaming into bone, and one or more of irrigation and suction capabilities.

Referring again to FIG. 1, an inventive illuminated translucent endoscopic probe 10 according to the disclosure is depicted in the context of spinal anatomy, namely the pedicle (P) of a vertebral body. As shown, the illuminated translucent endoscopic probe 10 includes a distal end 20 that terminates in a distal tapered tip 22, a proximal handle 60, a body 40 between the handle 60 and the tip 22. As depicted, the illuminated translucent endoscopic probe 10 includes adjacent the proximal handle 60 a manifold 30 with lines 52, 54 that can accommodate various inputs and outputs, such as one or more of fiber optic lighting, camera(s), fluid in and outflow, exhaust, and neuromonitoring/neurostimulation electrodes. It will be appreciated that the depicted manifold 30 and handle 60 are schematic in nature, and that other features, shapes, and configurations are possible and that the features as shown are in no way intended to be limiting with respect to the inventive illuminated translucent endoscopic probe 10.

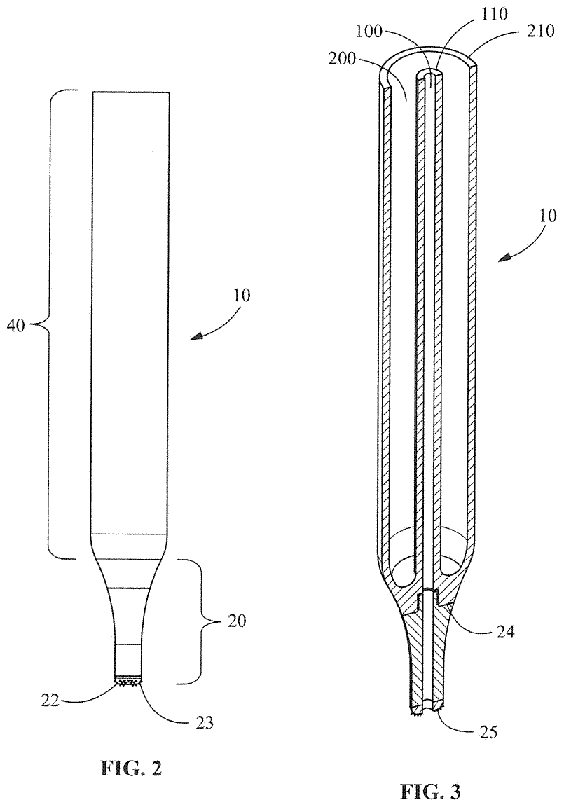

Referring now to FIG. 2-FIG. 6, alternate views of the distal end 20 and at least a portion of the body 40 of the illuminated translucent endoscopic probe 10 are shown. As shown in the cross-sectional view in FIG. 3, the illuminated translucent endoscopic probe 10 includes a central through channel 100 that extends from a proximal portion of the body 40 and through the distal tip 22. The channel 100 is shaped and sized to enable passage of a tool or instrument, for example a K-wire (Kirchner wire) or other guide line or wire that is customarily used in various surgical procedures. The illuminated translucent endoscopic probe 10 also includes within a portion of the body 40 but not within the distal end 20 of the illuminated translucent endoscopic probe 10, a peripheral void 200 that extends from a proximal portion of the body 40 and terminates within the body 40 at a point that is adjacent the proximal portion of the distal end 20.

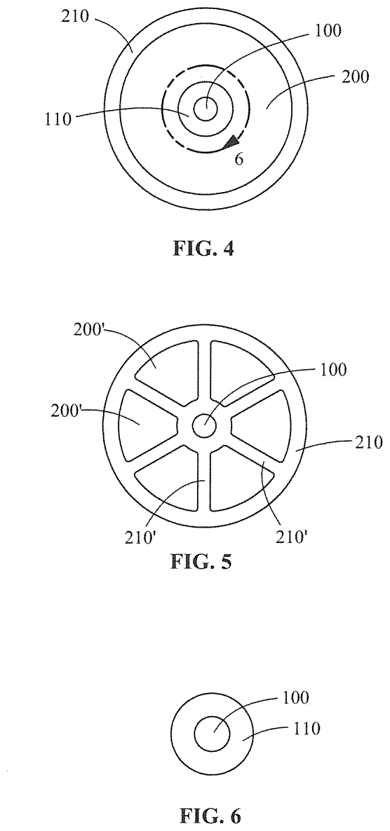

Referring again to FIG. 4-FIG. 6, alternate views in cross section across the longitudinal axis show the body 40 and the distal end 20 of the illuminated translucent endoscopic probe 10. Referring now to FIG. 6, the central through channel 100 is shown in a cross-sectional view of the distal end 20. Referring now to FIG. 4 and FIG. 5, alternate embodiments of the peripheral void 200 are shown. In FIG. 4, the peripheral void 200 is shown as a single circumferential peripheral void 200 that is defined between an inner wall 110 that bounds the central through channel 100, and a single outer wall 210, the peripheral void 200 extending from a proximal portion of the body 40 and ending adjacent the upper (proximal) portion of the distal end 20. As shown, the peripheral void 200 bottoms out into a curved shaped base, which in alternate embodiments may have a shape that is other than curved, and may be conical, planar or may have squared or radiused corners adjacent each of the inner wall 110 and outer wall 210.

Referring now to FIG. 5, an alternate embodiment of the peripheral void 200 is shown, the peripheral void 200 being formed by six discrete wedge-shaped channels 200′ and delineated by interior walls 210′. It will be appreciated that in yet other embodiments, the central through channel 100 may have a shape that is other than cylindrical and may be centrally located but not necessarily within the center per se of the illuminated translucent endoscopic probe 10. Further, one or more of the body 40 may have a shape that is other than cylindrical and the distal end 20 may likewise have a shape that is other than cylindrical and terminating in a tip that is cylindrical or conical. Further still, the peripheral void 200 may be formed of one or a plurality of discrete channels that are arranged other than concentrically with the central through channel 100. And the peripheral void 200 may be formed of one, two, three or more discrete channels. Thus, in an alternate example, the peripheral void 200 may be formed of two or more ovoid, circular or reniform (kidney) shaped channels. And, in yet another example, the peripheral void 200 may include two or more channels, one or more of which may terminate at a position along the length of the illuminated translucent endoscopic probe 10 that is different than the termination point of one or more other of the channels. And in the various embodiments, one or more of peripheral void 200 features may include properties of thickness and optical properties that may further define the peripheral void 200 and their termini.

In some alternate embodiments, the peripheral void 200 and/or one or more peripheral void channels 200 In some alternate embodiments, the peripheral void channels 200′ may be open on both ends (i.e., are through channels) where the distal end of the peripheral void 200 or void channels 200′ are open to an outside wall of the illuminated translucent endoscopic probe 10. According to some such embodiments, the illuminated translucent endoscopic probe 10 further comprises one or more additional components in the form of a lens or shield. According to such embodiments, the lens or shield is formed of a transparent polymeric, transparent glass or glass like material, while the portion of the body 40 that contains the peripheral void 200 may be formed of a transparent polymeric, transparent glass or glass like material or of another material that is not transparent.

In accordance with the various embodiments, the illuminated translucent endoscopic probe 10 includes the one or more of lighting and camera features deployed within the peripheral void 200. The relatively greater circumference of the body 40, particularly within the region containing the peripheral void 200, enables positioning of the lighting and cameras above and directed toward the distal end 20 of the illuminated translucent endoscopic probe 10. The number, arrangement, and other features of the lighting and camera components may be selected based on the optical properties of the material used to form the body 40 and distal end 20, and the lengths thereof, so as to provide the desired visualization of the distal tip 22 and surrounding tissue when the illuminated translucent endoscopic probe 10 is in use.

Referring again to FIG. 2 and FIG. 3, the distal end 20 includes a region that has a substantially uniform diameter through a portion of its length and terminates at the distal tip 22 with a tip face 23. In the depicted embodiment, the body 40 has a diameter that is greater than the diameter of the distal end 20 and the transition between the body 40 and distal end 20 are shown as a gradual taper. It will be appreciated that the nature of the transition between the body 40 and distal end 20 may be more abrupt or may be more gradual. In some embodiments, the illuminated translucent endoscopic probe 10 body 40 and distal end 20 are unitary and formed of the same material, such as a transparent polymeric, transparent glass or glass like material.

In other embodiments, only the distal end 20, or a portion of the body 40 and the distal end 20, are formed as separate part that is attachable to the body 40. In one such example, with reference to FIG. 3, the distal end 20 is engageable with a distal portion of the body 40 at a joint 24. According to yet other embodiments, the joint 24 may be located more proximally and at the juncture between the body 40 and the distal end 20 and below (i.e., not transecting) the peripheral void 200, or within the body 40 and transecting the peripheral void 200. As depicted, the optional feature of a removable distal end 20 via a joint 24 enables replacement where the distal tip 22 is either damaged, obscured due to abrasion, such as from bone, or replacement is desired to provide other features at the distal tip 22. In some embodiments the joint is a leur lock, and each of the body and the detachable tip include engageable leur taper and lock. In some embodiments, each of the detachable tip and the body include complimentary threading.

In some examples, the distal end 20 along at least a portion of its length has a diameter that is approximately the same as, or slightly smaller than, the diameter of a pedicle screw to be inserted in the hole formed with the illuminated translucent endoscopic probe 10, and will form an elongate hole having a uniform diameter for secure engagement with a screw inserted in the hole. In some embodiments, the illuminated translucent endoscopic probe 10 is adapted to penetrate hard cortical bone tissue, for example, the hard-cortical bony tissue of a vertebral pedicle to form a hole for reception of a pedicle screw. According to such embodiments, the tip has a hardness and configuration to act as a reamer to facilitate penetration of the probe through the hard-bony tissue. According to such embodiments, as shown in the drawings, the tip face 23 may be beveled, planar or pointed and comprise one or more of surface texturing features that enhance penetration of bone, such features selected from, for example, knurling, burrs, fluting, and teeth.

In some alternate embodiments, the distal tip 22 is adapted to engage with a removable cap or sleeve that may be formed of a metal or other material and that includes a leading tip that may be closed or cannulated and that includes a leading portion that includes structures or features that enhance penetration of bone. The removable cap or sleeve may be selectively engaged by a user to enable penetration of cortical bone and thereafter disengaged to permit a clear view of the distal end 20 of the illuminated translucent endoscopic probe 10 for visualization within tissue. It will be appreciated that in other embodiments, the tip face 23 may be essentially smooth and generally planar, beveled or pointed, and including edges that may be one of chamfered, beveled, planar or radiused.

In accordance with some embodiments, the probe is transparent along at least a portion of its length, the transparency sufficient to enable the passage of light from a proximally positioned light source to direct and provide illumination at the distal end. In accordance with some embodiments, the probe includes any one or more of a removable tip on its distal end, a distal cutting feature for reaming into bone, and one or more of irrigation and suction capabilities. In accordance with some embodiments, the probe includes a manifold with lines that can accommodate various inputs and outputs, such as one or more of fiber optic lighting, camera(s), fluid in and outflow, exhaust, and neuromonitoring/neurostimulation electrodes.

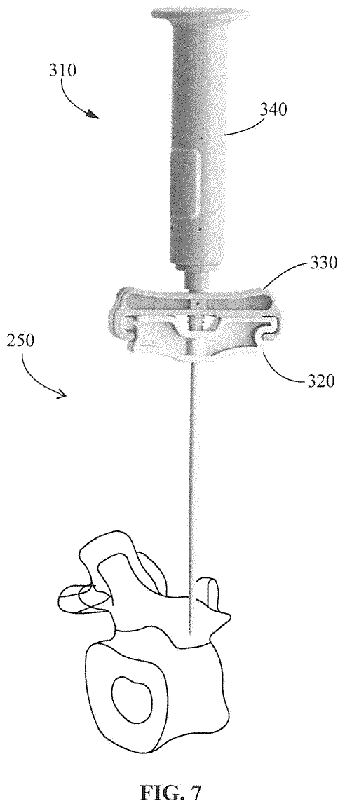

Referring again to the drawings, an embodiment of an inventive illuminated translucent endoscopic probe system 310 that includes a tissue penetrating probe 250 is shown in FIG. 7-FIG. 20. Key to the functionality of the penetrating translucent illuminated endoscopic probe system 310 is the rigidity of at least the outer access cannula 320 and the penetrating cannula 330 and the transparent and penetrating nature of the transparent driving head 350 which combination provides the mechanical features needed to penetrate and guide through solid tissue formed of soft, bony or connective tissue for real time visualization. These features constitute a significant and non-obvious improvement over probes in the art and enable for the first time the ability to view tissue at the distal tip of the probe, in real time, within generally solid tissue that is not defined by a lumen such as in the gastro intestinal and vascular systems.

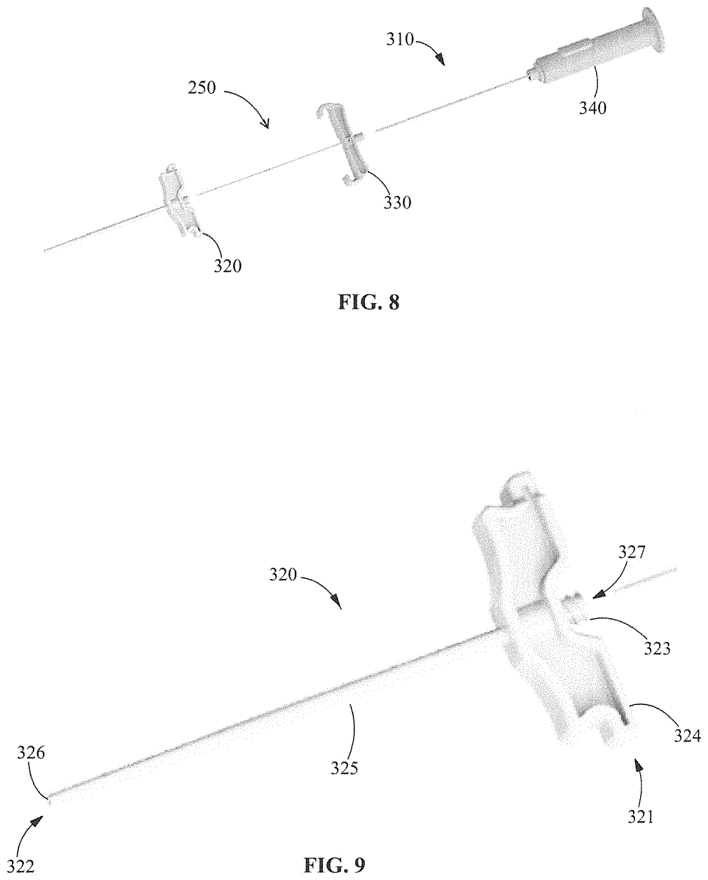

Referring now to FIG. 8, the tissue penetrating translucent illuminated endoscopic probe system 310 comprises a tissue penetrating probe 250 that comprises a rigid outer access cannula 320 and a rigid penetrating cannula 330 that has at its distal end 332 a transparent driving head 350, and is adapted to receive a scope component 340 which includes, in some embodiments, an scope component 340 with integrated camera and light. In accordance with the various embodiments, as is shown in FIG. 7 and FIG. 8, the rigid outer access cannula 320, rigid penetrating cannula 330, and scope component 340 with integrated camera and light components are inter-engagable along a common central elongate axis, the scope component 340 with integrated camera and light being slidably received within the rigid penetrating cannula 330, which is itself slidably received within the rigid outer access cannula 320.

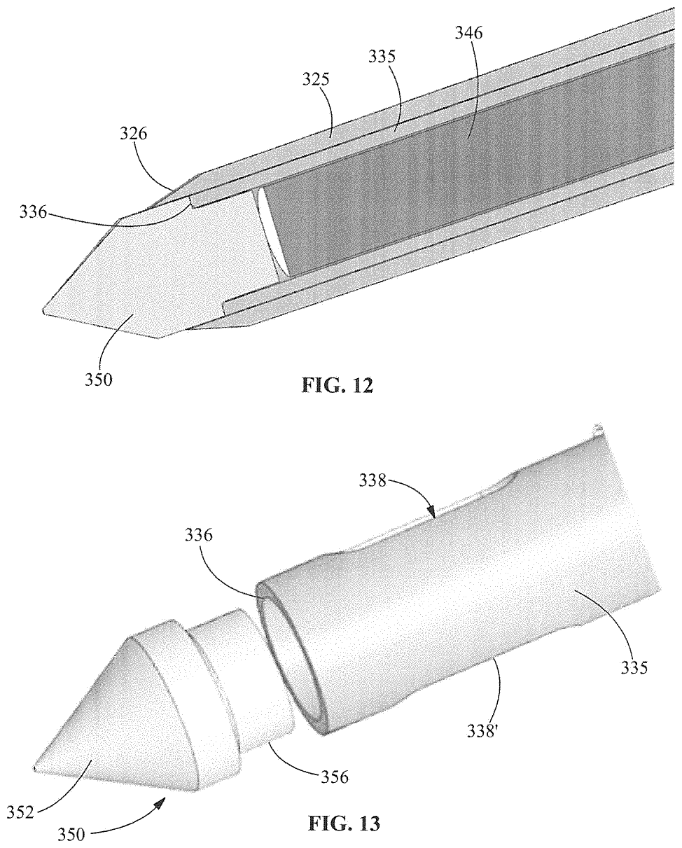

Referring to FIG. 12 and FIG. 13, the walls of the penetrating cannula 330 may be solid or may be fenestrated. Likewise, though not shown, the walls of the outer access cannula 320 may also be solid or fenestrated. In some embodiments the fenestrations may be slots 339, as depicted, or they may have other non-limiting shapes.

Referring now to FIG. 9-FIG. 11, as assembled, the tissue penetrating probe 250 comprises the outer access cannula 320 and a penetrating cannula 330 wherein the penetrating cannula 330 is slidably disposed within the through channel 327 of the outer access cannula 320. As assembled, the tissue penetrating translucent illuminated endoscopic probe system 310 includes the tissue penetrating probe 250 wherein the scope component 340 with integrated camera and light is slidably disposed within the through channel 337 of the penetrating cannula 330. As shown, the walls of the elongate rigid housing 325, 335 of the cannulas 320, 330 may be close in contact. In other embodiments, there may be spacing between the walls such that the diameter of the through channel 327 is closer to or much greater than the diameter of the elongate rigid housing 325, 335 of the penetrating cannula 330. This relationship is also true for the scope receiving channel 337 and its relationship dimensionally to the elongate scope housing 346 of the scope component 340 with integrated camera and light.

In various embodiments, the penetrating translucent illuminated endoscopic probe system 310 is adapted with suitable clearance to allow the passage of fluid (liquid or gas) between the elongate rigid housings 325, 335 of each component to enable delivery and removal of fluid at the distal end 322 of the penetrating translucent illuminated endoscopic probe system 310. In some embodiments, one or more of the penetrating cannula 330 and the scope component 340 with integrated camera and light may include on an outer wall along at least a portion of the length of their housings 335, 346 ribs or other shapes or textures (not shown) that allow fluidic movement and/or facilitate ease of slidable engagement and disengagement to prevent locking in the event of twisting or bending along the long axis of the assembled penetrating translucent illuminated endoscopic probe system 310 or exposure to moisture and the like when in use.

Referring to FIG. 11, the scope component 340 with integrated camera and light includes at its proximal end 341 a guide handle 345 that may have any configuration suited to gripping, including a cylindrical shape, as shown in the drawings, or some other ergonomically acceptable shape, such as for example a configuration like the handles 324, 334 depicted herein for the cannulas 320, 330. The guide handle 345 may include a grip or strike plate 343 as shown in the drawings, and an actuator 344 for one or more of actuating lighting, camera, or fluid. The guide handle 345 may in some embodiments house instrumentation needed for management of the heat and power to the camera and lighting may be contained therein and may further include wiring for attachment to power and video sources (not shown). At the distal tip of the body 346, the scope component 340 with integrated camera and light includes a lighting array and a camera. As shown in the drawings, in the depicted embodiment, the lighting array 348 is positioned around the periphery of the distal tip 347 and a camera 349 is positioned at the center. Of course, it will be appreciated that the drawings depict one possible organization of the lighting and camera of the scope component 340, for example, employing a commercially available product such as the Medigus LED probe, which is an integrated camera and illumination device available from Medigus, Ltd. of Omer, Israel. The Medigus LED probe is a 1.8/2.0 mm diameter rigid endoscope which includes a 1.2 mm camera in the distal tip of the device. It is equipped with high quality 100°/140° field of view (FOV) optics and a large LED located in the handle of the device. The device has a stainless-steel shaft and illumination is led through the shaft towards the distal tip of the device where the camera is located via fiber-for-illumination. The LED is powered by the video processor and, therefore, no additional peripherals are required other than a monitor. The camera used with this system has a diameter of only 1.2 mm and a length of only 5 mm. It has high quality 100° FOV optics and a shielded camera cable with a metal connector as well as a video processor. It will be appreciated that other configurations for lighting and camera are possible.

Referring again to FIG. 8-FIG. 10, each of the outer access cannula 320 and the penetrating cannula 330 is secured to a handle 324, 334. The handle 324, 334 includes a fluid manifold port 323, 333. The handle 324, 334 is ergonomically shaped to comfortably fit into a surgeon's or other user's hand. The handle 324, 334 is shaped to conform to an individual's palm. The shape of the handle 324, 334 is designed to be enclosed and grasped by the fingers to assist in the control of the cannula 320, 330. The handle 324, 334 can be used to stabilize the cannula 320, 330 for attachment of a fluid suction or removal conduit to the fluid manifold port 323, 333, or to drive the cannula 320, 330 into tissue. In some embodiments, the tissue penetrating probe 250 can be driven through the bone and/or soft tissue by direct manipulation of the handle 324, 334 by the user applying only manual pressure. As depicted, the handles 324, 334 are adapted for engagement and locking. It will be appreciated that in some embodiments, the engagement and locking features between the outer and inner cannula may be by means at other than the proximal end 321. For example, each may comprise complimentary features at their distal ends 322, 332 for engagement such as by snap fitting, twist locking, and threaded engagement.

In other embodiments, the penetrating translucent illuminated endoscopic probe system 310 may be fully assembled to enable application of a hammer or other instrument on the grip/strike plate 343 of the scope component 340 with integrated camera and light 340. In yet other embodiments, a driver attachment (not shown) may be engaged with the tissue penetrating probe 250 components, the attachment including a grip/strike plate 343 feature to facilitate driving the instrument with force other than simple manual pressure or rotation. The outer access cannula 320 is suitable for passage of a Kirschner wire, K-wire or the like into the through channel 327 to a surgical site. In some embodiments, the outer access cannula 320 is suitable for passage of a screw for implantation into bone, or another instrument for manipulation of tissue within the surgical site.

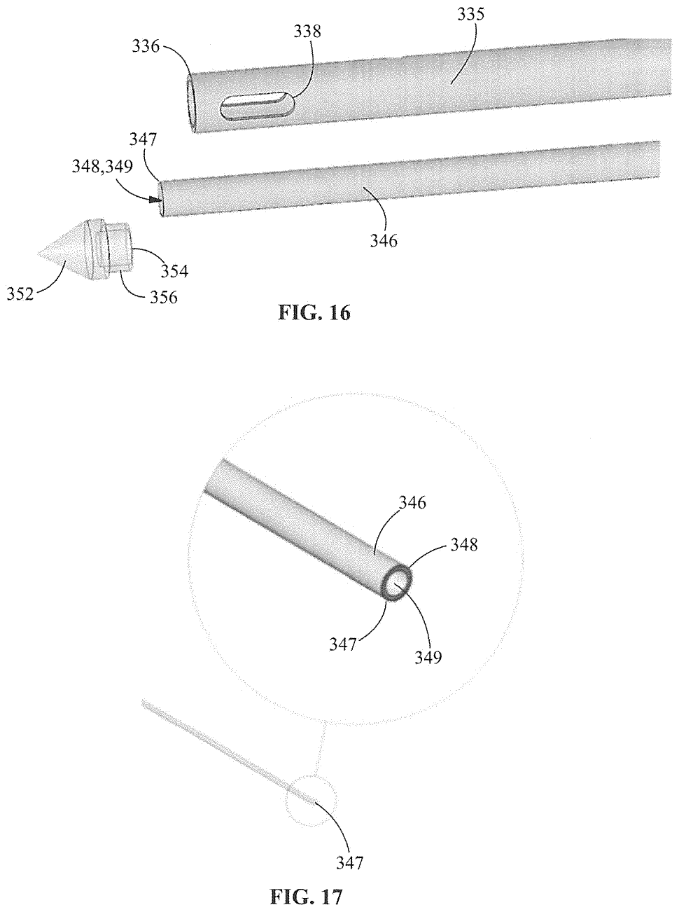

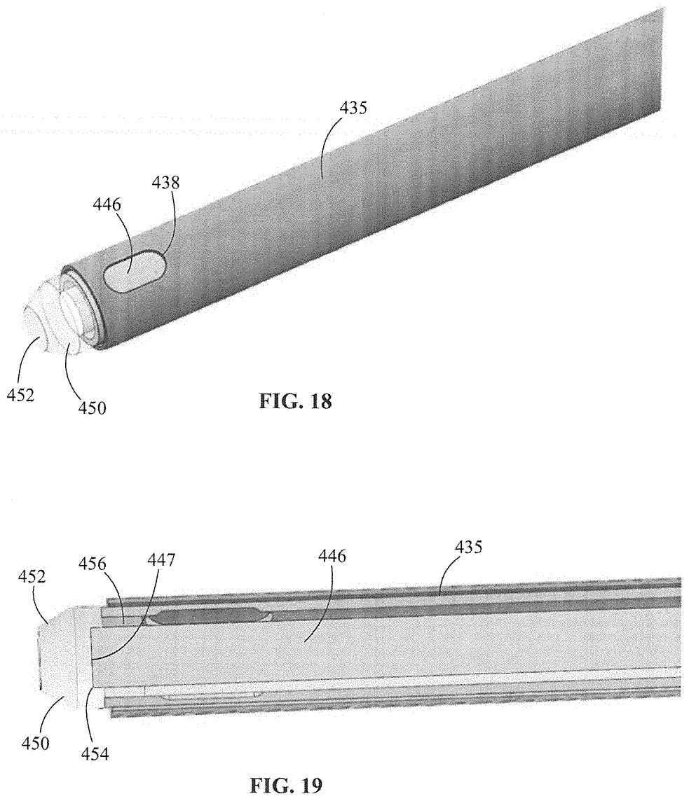

Referring now to FIG. 12, the penetrating cannula 330 is specifically adapted at its distal end 332 for engagement with a transparent driving head 350. In accordance with the inventive embodiments, this transparent driving head 350 has a contoured penetrating tip 352 and is formed of a material that has mechanical properties that enable it to resist one or more of fracture, cracking, and crazing while providing translucency and a degree of optical transparency. Examples of material suitable for this use include glasses, translucent metal ceramics, and polymers. Referring now to FIG. 12-17 and FIG. 18-20, the transparent driving head 350 may have a contoured penetrating tip 352 that is one of conical contoured tip 358 as shown for example in FIG. 16, or frustoconical contoured tip 458, as shown for example in FIG. 18, comprising a face that is one of planar, convex or concave. Further the transparent driving head 350 may have a solid body 357, or cannulated body 457 that is either solid or cannulated and thus has an open end. In some such embodiments, the transparent driving head 350 may comprise a scope seating recess 354, 454 for receiving the scope component 340 with integrated camera and light distal end 342. In the embodiments depicted, for example in FIG. 16, the transparent driving head 350 has a solid body 357 with a locking body 356 that has a generally planer face that opposes the distal end 342 of the scope component 340 with integrated camera and light. It will be appreciated that there are other possible configurations for the locking body 356 at its interface with a scope component 340 with integrated camera and light when the scope is inserted in the scope receiving channel 337 of the penetrating cannula 330 adjacent the engaged transparent driving head 350.

In some examples, the elongate rigid housing 325 of the outer access cannula 320 along at least a portion of its length has a diameter that is approximately the same as, or slightly smaller than, the diameter of a screw, for example a pedicle screw, to be inserted into the hole formed with the tissue penetrating translucent illuminated endoscopic probe system 310, and will form an elongate hole having a uniform diameter for secure engagement with a screw inserted into the hole. In some embodiments, the tissue penetrating translucent illuminated endoscopic probe system 310 is adapted to penetrate hard cortical bone tissue, for example, the hard-cortical bony tissue of a vertebral pedicle to form a hole for reception of a pedicle screw. According to such embodiments, the transparent driving head 350 has a hardness and configuration to act as a reamer to facilitate penetration of the probe through the hard-bony tissue.

In accordance with various embodiments, the tissue penetrating translucent illuminated endoscopic probe system may further include any one or more of functions including suction, fluidic (liquidous and air) irrigation, ventilation. In some embodiments, one or more of such features is delivered via attachment to a manifold at the proximal end of the tissue penetrating translucent illuminated endoscopic probe system and such features may be provided, for example but not limited to flow through a central through channel of an outer access cannula.

The tissue penetrating translucent illuminated endoscopic probe of the invention provides the surgeon with an illuminated, direct visual indication of the exact location of the probe and provides for flushing body fluids and debris away from the area being treated, whereby the hole can be formed with accuracy and precision.

In accordance with the various embodiments herein, one or more components may be formed of a transparent material. In some embodiments, the transparent material may comprises conventionally known polymeric materials suitable for medical applications and having general transparency. In some embodiments, particular with respect to hard tip portions suitable for penetration of bone and tissue, the transparent material may be a transparent aluminum nitride. For example, the material ALON® or Aluminum Oxynitride is a transparent advanced ceramic that is polycrystalline (made from powder) with a cubic spinel crystal structure. This material may be obtained commercially from Surmet of Burlington, Mass. The properties of this material (as described by Surmet at URL: surmet.com/technology/alon-optical-ceramics/), are provided in Table 1.

TABLE 1

| | |
|---|---|
| Composition: | Al_{23-1/3X}O_{27+X}N_{5-X} |
| Grain Size (typical): | 150-250 microns |
| Structure: | Cubic, Spinel |
| Density: | 3.696-3.691 g/cc |
| Form: | Polycrystalline |
| Melting Point: | 2150° C. |
| Young's Modulus: | 323 GPa |
| Hardness: | 1850 kg/mm² (Knoop Indent, 200 g) |
| Fracture Toughness: | 2.0-2.9 MPa-m^{1/2} |
| Flexure Strength: | 380-700 MPa |
| Thermal Conductivity | 13 W/mK |
| Transmission Limits: | 0.22 to 6 microns |

As used herein, the singular forms “a,” “an,” and “the” are intended to include the plural forms as well, unless the context clearly indicates otherwise. The term “proximal” as used in connection with any object refers to the portion of the object that is closest to the operator of the object (or some other stated reference point), and the term “distal” refers to the portion of the object that is farthest from the operator of the object (or some other stated reference point). The term “operator” means and refers to any professional or paraprofessional who delivers clinical care to a medical patient, particularly in connection with the delivery of care.

Anatomical references as used herein are intended to have the standard meaning for such terms as understood in the medical community. For example, the application may include reference to the following terms: “cephalad,” “cranial” and “superior” indicate a direction toward the head, and the terms “caudad” and “inferior” indicate a direction toward the feet. Likewise, the terms “dorsal” and “posterior” indicate a direction toward the back, and the terms “ventral” and “anterior” indicate a direction toward the front. And the term “lateral” indicates a direction toward a side of the patient. The term “medial” indicates a direction toward the mid line of the patient, and away from the side, the term “ipsalateral” indicates a direction toward a side that is proximal to the operator or the object being referenced, and the term “contralateral” indicates a direction toward a side that is distal to the operator or the object being referenced.

Unless otherwise indicated, all numbers expressing quantities, properties, and so forth as used in the specification, drawings and claims are to be understood as being modified in all instances by the term “about.” Accordingly, unless otherwise indicated, the numerical properties set forth in the specification and claims are approximations that may vary depending on the suitable properties desired in embodiments of the present invention. Notwithstanding that the numerical ranges and parameters setting forth the broad scope of the general inventive concepts are approximations, the numerical values set forth in the specific examples are reported as precisely as possible. Any numerical values, however, inherently contain certain errors necessarily resulting from error found in their respective measurements.

References to visualization using radiography as may be described in the exemplary techniques herein are merely representative of the options for the operator to visualize the surgical field and the patient in one of many available modalities. It will be understood by one of ordinary skill in the art that alternate devices and alternate modalities of visualization may be employed depending on the availability in the operating room, the preferences of the operator and other factors relating to exposure limits. While confirmation of instrument placement in the course of the technique is appropriate, the frequency and timing relative to the sequence of steps in the technique may be varied and the description herein is not intended to be limiting. Accordingly, more or fewer images, from more or fewer perspectives, may be collected.

One of ordinary skill will appreciate that references to positions in the body are merely representative for a particular surgical approach. Further, all references herein are made in the context of the representative images shown in the drawings. Fewer or additional instruments, including generic instruments, may be used according to the preference of the operator. Moreover, references herein to specific instruments are not intended to be limiting in terms of the options for use of other instruments where generic options are available, or according to the preference of the operator.

While the disclosed embodiments have been described and depicted in the drawings in the context of the human spine, it should be understood by one of ordinary skill that all or various aspects of the embodiments hereof may be used in in connection with other species and within any species on other parts of the body where deep access within the tissue is desirable.

While various inventive aspects, concepts and features of the general inventive concepts are described and illustrated herein in the context of various exemplary embodiments, these various aspects, concepts and features may be used in many alternative embodiments, either individually or in various combinations and sub-combinations thereof. Unless expressly excluded herein all such combinations and sub-combinations are intended to be within the scope of the general inventive concepts. Still further, while various alternative embodiments as to the various aspects, concepts and features of the inventions (such as alternative materials, structures, configurations, methods, devices and components, alternatives as to form, fit and function, and so on) may be described herein, such descriptions are not intended to be a complete or exhaustive list of available alternative embodiments, whether presently known or later developed.

Those skilled in the art may readily adopt one or more of the inventive aspects, concepts and features into additional embodiments and uses within the scope of the general inventive concepts, even if such embodiments are not expressly disclosed herein. Additionally, even though some features, concepts and aspects of the inventions may be described herein as being a preferred arrangement or method, such description is not intended to suggest that such feature is required or necessary unless expressly so stated. Still further, exemplary or representative values and ranges may be included to assist in understanding the present disclosure; however, such values and ranges are not to be construed in a limiting sense and are intended to be critical values or ranges only if so expressly stated.

Moreover, while various aspects, features and concepts may be expressly identified herein as being inventive or forming part of an invention, such identification is not intended to be exclusive, but rather there may be inventive aspects, concepts and features that are fully described herein without being expressly identified as such or as part of a specific invention. Descriptions of exemplary methods or processes are not limited to inclusion of all steps as being required in all cases, nor is the order that the steps are presented to be construed as required or necessary unless expressly so stated.

1. An illuminated translucent endoscopic probe comprising:
(a) a tissue penetrating probe comprising rigid cannula components that are inter-engagable along a common central elongate axis, the probe comprising:
(i) a rigid outer access cannula; and
(ii) a rigid penetrating cannula that comprises at its distal end a transparent driving head, and
(b) a scope component;
wherein the rigid outer access cannula includes an inner through channel bounded by a distal tip and a proximal opening and adapted to slidably receive the rigid penetrating cannula, and wherein the rigid penetrating cannula includes an inner through channel bounded by the distal transparent driving head and a proximal opening and adapted to slidably receive the scope component.

2. The illuminated translucent endoscopic probe according to claim 1, wherein each of the components of the tissue penetrating probe comprises a proximal handle for cooperation with the hand of a user to aid in controlling the component.

3. The illuminated translucent endoscopic probe according to claim 1, wherein one or more of the elongate body of the rigid penetrating cannula and the transparent driving head is formed of a transparent material selected from polymeric, glass, and metal-ceramic glass-like material.

4. The illuminated translucent endoscopic probe according to claim 1, wherein the elongate body of the rigid penetrating cannula and the transparent driving head are not unitary and are releasable coupled.

5. The illuminated translucent endoscopic probe according to claim 1, wherein the transparent driving head is formed of a transparent material selected from polymeric, glass, and metal-ceramic glass-like material.

6. The illuminated translucent endoscopic probe according to claim 1, wherein each of the elongate body of the rigid penetrating cannula and the transparent driving head is formed of a transparent material selected from polymeric, glass, and metal-ceramic glass-like material.

7. The illuminated translucent endoscopic probe according to claim 6, wherein the elongate body of the rigid penetrating cannula and the transparent driving head is unitary.

8. The illuminated translucent endoscopic probe according to claim 3, wherein the transparent material is a metal-ceramic glass-like material.

9. The illuminated translucent endoscopic probe according to claim 8, wherein the transparent material is aluminum oxynitride.

10. An illuminated translucent endoscopic probe comprising:
a handle for cooperation with the hand of a user to aid in controlling the probe;
an body having a proximal end that is engageable with the handle and having a longitudinal axis that extends from the handle to a distal end, the body comprising
a central through channel that extends through the body from its engagement with the handle and to the distal end;
a peripheral void that at least partially surrounds the central through channel and extends to and terminates adjacent an upper portion of the distal end;
wherein the distal end of the body and at least a portion of the body that is proximal to the distal end are formed of a transparent material selected from polymeric, glass, and metal-ceramic glass-like material.

11. The illuminated translucent endoscopic probe according to claim 10, wherein a tip has a hardness and configuration adapted to be pushed through bone.

12. The illuminated translucent endoscopic probe according to claim 10, wherein the body is cylindrical, and the distal end is cylindrical and terminates in a tip that frustoconical.

13. The illuminated translucent endoscopic probe according to claim 12, the probe further comprising lighting and least one camera positioned proximally to enable real time visualization as the probe is moved forward in tissue.

14. The illuminated translucent endoscopic probe according to claim 11, wherein the central through channel has a shape that is cylindrical.

15. The illuminated translucent endoscopic probe according to claim 11, wherein the peripheral void comprises a single circumferential void channel that is defined between an inner wall that bounds the central through channel, and an outer wall.

16. The illuminated translucent endoscopic probe according to claim 11, wherein the peripheral void bottoms out into a curved shaped base, which in alternate embodiments may have a shape that is other than curved, and may be conical, planar or may have squared or radiused corners adjacent each of the inner wall and outer wall.

17. The illuminated translucent endoscopic probe according to claim 11, wherein the peripheral void is formed by two or more discrete peripheral void channels each delineated by interior walls.

18. The illuminated translucent endoscopic probe according to claim 11, wherein the peripheral void comprises one or a plurality of discrete peripheral void channels that are arranged other than concentrically with the central through channel.

19. The illuminated translucent endoscopic probe according to claim 11, wherein the peripheral void comprises two, three or more discrete peripheral void channels, and wherein the peripheral void comprises two or more ovoid, circular or reniform shaped peripheral void channels.

20. The illuminated translucent endoscopic probe according to claim 11, wherein the transparent material comprises aluminum oxynitride.
